SEQUENCE LISTING (2 sequences)

SEQ ID NO: 1 - DNA, 20 bp, Artificial Sequence
  5′ primer
attcctggctttgtgctctg

SEQ ID NO: 2 - DNA, 20 bp, Artificial Sequence
  3′ primer
tggcttcttggctctcttgt

(19) United States
(12) Patent Application Publication
(10) Pub. No.: US 2016/0130662 A1
(43) Pub. Date: May 12, 2016

(54) NOVEL GENOMIC MARKER OF METASTATIC PROSTATE CANCER

(71) Applicant: THE JOHNS HOPKINS UNIVERSITY, Baltimore, MD (US)

(72) Inventors: Paula J. HURLEY, Perry Hall, MD (US); Edward M. SCHAEFFER, Sparks, MD (US)

(21) Appl. No.: 14/899,352

(22) PCT Filed: Jun. 17, 2014
(86) PCT No.: PCT/US2014/042629

§ 371 Date: Dec. 17, 2015

Related U.S. Application Data
(60) Provisional application No. 61/835,752, filed on Jun. 17, 2013.

(51) Int. Cl. C12Q 1/68

(52) U.S. Cl. CPC C12Q 1/6886; C12Q 2600/118; C12Q 2600/156

(57) ABSTRACT

The present invention relates to the field of cancer. More specifically, the present invention provides methods and compositions useful for assessing prostate cancer in a patient. In a specific embodiment, a method for predicting metastasis in a prostate cancer patient comprises the steps of (a) genotyping the ASPN D repeat domain length in both alleles of the patient using a polymerase chain reaction; (b) predicting metastasis in the patient if the ASPND repeat domain length is 13 in one allele and 14 in the other allele or have at least one allele with 14 D repeats as compared to other common allelic genotypes; and (c) predicting no metastasis in the patient if the ASPN D repeat domain length is 13 in both alleles as compared to other allelic genotypes.

### CROSS-REFERENCE TO RELATED APPLICATIONS

This application claims the benefit of U.S. Provisional Application No. 61/835,752, filed Jun. 17, 2013, which is incorporated herein by reference in its entirety.

### STATEMENT OF GOVERNMENTAL INTEREST

This invention was made with government support under grant no. W81XWH-10-2-0056 awarded by Department of Defense. The government has certain rights in the invention.

### FIELD OF THE INVENTION

The present invention relates to the field of cancer. More specifically, the present invention provides methods and compositions useful for assessing prostate cancer in a patient.

### INCORPORATION-BY-REFERENCE OF MATERIAL SUBMITTED ELECTRONICALLY

This application contains a sequence listing. It has been submitted electronically via EFS-Web as an ASCII text file entitled “P12578-01_Sequence_Listing_ST25.txt.” The sequence listing is 616 bytes in size, and was created on Jun. 17, 2014. It is hereby incorporated by reference in its entirety.

### BACKGROUND OF THE INVENTION

One in six men will be diagnosed with prostate cancer; yet not all of these men will progress to lethal disease. Development of genomic prognostic biomarkers for early differentiation between indolent and aggressive prostate cancer remains a clinical challenge. We and others have shown that early prostate development is an excellent model system to examine molecular processes driving metastatic prostate cancer (1-3). We previously reported that Asporin (Aspn), a member of a small leucine-rich proteoglycan (SLRP) family of extracellular proteins was one of the most highly enriched genes during early invasive phases of prostate development and during cancer progression (1). ASPN expression correlates with multiple diseases including cancer (4-11). While elevated ASPN expression has been found in prostate cancer reactive stroma (8) and in breast carcinoma cells (4, 9), a biologic role for ASPN in cancer is not known.

In addition to expression, a genetic association exists between variation at the ASPN locus and multiple disease states (12-17). ASPN contains an aspartic acid (D) repeat domain, and polymorphisms in the length of this allelic repeat have been associated with susceptibility to bone and joint diseases including osteoarthritis (12, 13, 15, 16), lumbar-disc degeneration (17), and developmental dysplasia of the hip (18) in Asian populations. The ASPN D repeat domain has been shown to bind calcium (19) and to have a role in osteoblast-driven collagen mineralization (19). Over 10 polymorphisms in the length of the D repeat have been identified with the most common variants ranging from 12-16 aspartate residues (13). Polymorphisms in the ASPN D repeat domain length have been shown to differentially inhibit Transforming Growth Factor Beta 1 (TGFβ1) induced gene expression (13). While functional differences between these polymorphisms are not known, ASPN D repeat length of 14 (ASPN D14) has been shown to be associated with osteoarthritis (12, 13, 15, 16).

### SUMMARY OF THE INVENTION

The present invention is based, at least in part, on the discovery of two common germline variants of a single gene that are associated (one positively and one negatively) with metastatic prostate cancer. A genetic test based on these findings has clinical utility for therapeutic decisions pertaining to Active Surveillance, surgery, and multi-modal therapies.

Prostate cancer remains the second leading cause of cancer death among US men due to a subset of cancers that progress to metastatic disease. We hypothesized that germline variants in the aspartic acid (D) repeat domain of ASPORIN (ASPN) may be associated with metastatic prostate cancer progression.

Multivariable analyses demonstrated that men with an ASPN D repeat domain length of 13 in both alleles (13/13) were significantly less likely to progress to metastatic disease (Hazard Ratio [HR]: 0.43, P=0.038) than men with other common allelic genotypes. In contrast, multivariable analyses demonstrated that men who have an ASPN D repeat domain length of 13 in one allele and 14 in the other allele (13/14) or who have at least one allele with 14 D repeats (14/x) were significantly more likely to have lymph node metastases at surgery (Odds Ratio [OR] for 13/14: 2.62, P=0.006 and OR for 14/x: 1.95, P=0.033) than men with other common allelic genotypes.

Accordingly, in one aspect, the present invention provides methods for determining metastatic prostate cancer progression. In one embodiment, a method for determining a likelihood of prostate cancer recurrence in a patient following prostectomy comprises the steps of (a) obtaining a biological sample from the patient; (b) subjecting the sample to an assay for genotyping the aspartic acid (D) repeat polymorphism located in the N-terminal region of the Asporin (ASPN) gene; (c) determining that prostate cancer is less likely to recur if the ASPN D repeat domain length is 13 in both alleles as compared to other allelic genotypes; and (d) determining that prostate cancer is more likely to recur if the ASPN D repeat domain length is 13 in one allele and 14 in the other allele or have at least one allele with 14 D repeats as compared to other allelic genotypes.

In another embodiment, a method for predicting metastasis in prostate cancer patient comprises the steps of (a) obtaining a biological sample from the patient; (b) subjecting the sample to an assay for genotyping the aspartic acid (D) repeat polymorphism located in the N-terminal region of the Asporin (ASPN) gene; (c) determining that metastasis is less likely to occur if the ASPN D repeat domain length is 13 in both alleles as compared to other allelic genotypes; and (d) determining that metastasis is more likely to recur if the ASPN D repeat domain length is 13 in one allele and 14 in the other allele or have at least one allele with 14 D repeats as compared to other allelic genotypes.

The present invention also provides a method for identifying prostate cancer lesions with metastatic potential in a patient comprising the steps of (a) obtaining a biological sample from the patient; (b) subjecting the sample to an assay for genotyping the aspartic acid (D) repeat polymorphism located in the N-terminal region of the Asporin (ASPN) gene; (c) determining that the prostate cancer lesions have lower metastatic potential if the ASPN D repeat domain length is 13 in both alleles as compared to other allelic genotypes; and (d) determining that the prostate cancer lesions have higher metastatic potential if the ASPN D repeat domain length is 13 in one allele and 14 in the other allele or have at least one allele with 14 D repeats as compared to other allelic genotypes.

In certain embodiments of the methods of the present invention, the obtaining the biological sample step comprises obtaining tissue for genomic DNA isolation. In addition, in some embodiments, the performing an assay step comprises performing a polymerase chain reaction. In a specific embodiment, the polymerase chain reaction is performed using the primer described in SEQ ID NO:1 and SEQ ID NO:2.

In a further embodiment, a method for predicting metastasis in a prostate cancer patient comprises the steps of (a) genotyping the ASPN D repeat domain length in both alleles of the patient using a polymerase chain reaction; (b) predicting metastasis in the patient if the ASPND repeat domain length is 13 in one allele and 14 in the other allele or have at least one allele with 14 D repeats as compared to other common allelic genotypes; and (c) predicting no metastasis in the patient if the ASPN D repeat domain length is 13 in both alleles as compared to other allelic genotypes.

### BRIEF DESCRIPTION OF THE FIGURES

FIG. 1. Distribution of ASPN D Repeat Domain Length. ASPN D repeat domain length was determined by STR analysis in cohort of men (n=912) surgically treated at JHH for prostate cancer. The distribution of ASPN D repeat length ranged from 10 to 19 repeats, with 13, 15, and 14 repeats being the most common.

FIG. 2. Survival Estimates for ASPN D13/13 and ASPN D13/14. ASPN D13/13 is significantly prognostic of a reduced risk of metastatic prostate cancer (a), while ASPN D13/14 is significantly prognostic of metastatic prostate cancer. Kaplan-Meier curves for (a) ASPN D13/13 (n=201; Log-rank P=0.04) and (b) ASPN D13/14 (n=124; Log-rank P=0.02 with metastasis as an endpoint.

FIG. 3. Summary of JHU Cohort Characteristics and Outcomes on Follow-Up.

FIG. 4. Summary of JHU Cohort ASPN D Repeat Length Genotypes.

FIG. 5. Summary of ASPN D Repeat Length Genotypes with Clinical Data.

FIG. 6. Summary of ASPN D Repeat Length Genotypes with Clinical Outcomes.

FIG. 7. Comparison of Clinical Data Between ASPN D13/13 with ASPN D13/14.

FIG. 8. Comparison of Clinical Outcomes Between ASPN D13/13 with ASPN D13/14.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

Prostate cancer remains the second leading cause of cancer death among US men due to a subset of cancers that progress to metastatic disease. We hypothesized that germline variants in the aspartic acid (D) repeat domain of ASPORIN (ASPN) may be associated with metastatic prostate cancer progression.

This study analyzed ASPN D repeat domain length in a cohort of 912 patients treated with radical prostatectomy for prostate cancer at JHU. Multivariable analyses demonstrated that men with an ASPN D repeat domain length of 13 in both alleles (13/13) were significantly less likely to progress to metastatic disease (Hazard Ratio [HR]: 0.43, P=0.038) than men with other common allelic genotypes. In contrast, multivariable analyses demonstrated that men who have an ASPN D repeat domain length of 13 in one allele and 14 in the other allele (13/14) or who have at least one allele with 14 D repeats (14/x) were significantly more likely to have lymph node metastases at surgery (Odds Ratio [OR] for 13/14: 2.62, P=0.006 and OR for 14/x: 1.95, P=0.033) than men with other common allelic genotypes.

While homozygosity for the ASPN D13 variant is significantly prognostic of a reduced risk of metastatic recurrence, heterozygous ASPN D13/14 is significantly associated with an increased risk of lymph node metastases at surgery. These are the first germline variants reported that differentiate between indolent and aggressive prostate cancer. Not only do these findings have potential clinical utility for Active Surveillance decisions, they also provide mechanistic insights into prostate cancer progression.

### Materials and Methods

Study Population.

The study population is a hospital-based case population at the Johns Hopkins Hospital (JHH). All participants provided written informed consent. The protocol and consent documents were approved by the JHU Institutional Review Board. Prostate cancer cases were 100% men of non-Hispanic European descent (by self report) who underwent radical prostatectomy for treatment of prostate cancer at JHH from February 1993 through November 2012. Patients treated prior to the PSA era (1992 and prior) and those who received neoadjuvant hormonal treatments were excluded from the study. Considering the aforementioned criteria, 1000 cases were selected at random for genomic analysis, of which 912 had adequate DNA for genotyping. Radical prostatectomy specimens were processed as previously described (20). Each tumor was graded using the Gleason scoring system and staged using the TMN (tumor-node-metastasis) system. Patients who met any of the following criteria were considered as having more aggressive disease: pathologic Gleason score of 7 or higher, stage pT3 or higher, N+, or M1 (21). Clinical outcome data included biochemical recurrence (BCR) (defined as a post-operative Prostate Specific Antigen (PSA)≧0.2 ng/ml), distant metastasis (defined as clinical or radiographic spread of disease to extra-pelvic lymph nodes, bones, or viscera), and prostate-cancer specific mortality (PCSM).

Genomic DNA Isolation.

Tissue for genomic DNA isolation was taken from seminal vesicles (SV) at the time of radical prostatectomy. SV tissue was suspended in 12 ml Suspension buffer (20 mM Tris; 25 mM EDTA; 100 mM NaCl)+1 ml?% SDS+60 μl Proteinase K solution (20 mg/mL), inverted twice, and then incubated overnight at 50° C. The next day, RNA was digested by adding 60 μl RNase A Solution (Qiagen) and incubating at 37° C. for 1 hour. Proteins were precipitated in 4 ml Protein Precipitation Solution (Promega) on ice for 20 minutes and then centrifuged at 2,000 g for 10 minutes. DNA was extracted from the supernatant with 30 ml 100% Isopropanol, centrifuged 2,000 g for 5 minutes, and then washed with 70% Ethanol followed by centrifugation.

Genotyping of the D-Repeat Polymorphism.

The D repeat polymorphism located in the N-terminal region of the ASPN gene was polymerase chain amplified (PCR) amplified using 5′ primer 6-FAM-ATTCCTGGCTTTGTGCTCTG (SEQ ID NO:1) and 3′ primer TGGCTTCTTGGCTCTCTTGT (SEQ ID NO:2). Primers were designed using Oligo software. Other primers can be designed using methods and software known to those of ordinary skill in the art. Reactions were carried out in 10 μL consisting of 30 ng DNA, 0.125 μM primers, 0.6 mM dNTPs (Continental Lab Products), 10 mM Tris-HCl pH8.3, 50 mM KCL, 1.5 mM MgCl, and 0.6 units of Taq Gold DNA polymerase (Perkin Elmer). Amplification was performed in a Veriti Thermal Cycler (Applied Biosystems Inc.) for an initial denaturation of 12 minutes at 94° C. followed by 40 cycles of 94° C. for 20 sec, 58° C. for 20 sec, 72° C. for 30 sec, and a final 10 minute elongation at 72° C. The PCR products were electrophoresed on an ABI 3730xl DNA Analyzer (Applied Biosystems Inc.). Data was collected and analyzed with GeneMapper software (Applied Biosystems Inc.) that calculates fragment length in reference to an internal lane standard (LIZ500). Three homozygous samples of different repeat length were confirmed by Sanger sequencing. PCR products were sequenced using fluorescent dideoxy terminator method of cycle sequencing. Reactions were run on a 3730xl DNA Analyzer (Applied Biosystems Division) following Applied Biosystems protocols. Sequence data was analyzed using Sequencer Software (Gene Codes).

Statistical Analysis.

Characteristics of patient groups defined by distinct ASPN variations were compared. Means of continuous variables were compared by t-tests. Medians of non-normally distributed variables were compared by Wilcoxon-Mann-Whitney rank-sum tests. Proportions were compared by t-tests. Unviariate and multivariable logistic regression analysis was performed in order to test strengths of association with ASPN variations and pathologic outcomes: organ-confined disease, extracapsular extension, seminal vesical invasion, and lymph node metastasis. Similarly, univariate and multivariable Cox proportional hazards models were used to test the associations of ASPN variations with risk of subsequent oncologic outcomes: BCR, metastasis, and PCSM. Survival estimates were derived from Kaplan-Meier life tables. Statistics were computed with Stata 11.0 (StataCorp).

### Results

JHU Cohort Characteristics.

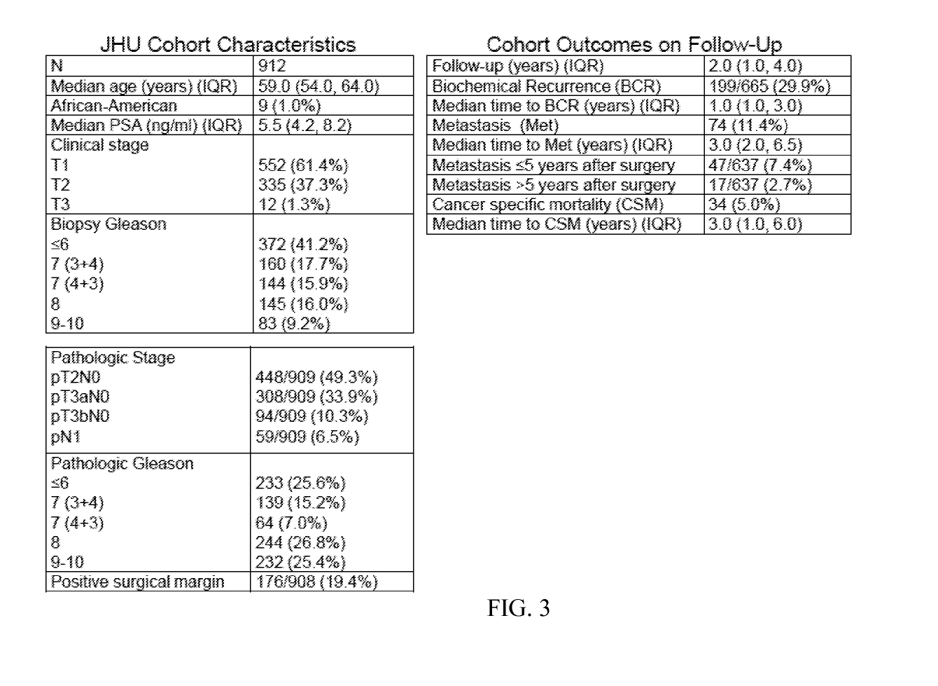

The study population included 912 men who underwent radical prostatectomy and pelvic lymphadenectomy for clinically localized prostate cancer at JHU. The median age at radical prostatectomy for treatment of prostate cancer was 59.0 years, with an average of 2.0 years follow up time after surgery (FIG. 3). To eliminate any confounding variables due to racial differences in allelic length distribution, all participants were Caucasian. Most men (93.5%) had organ confined prostate cancer at surgery and the median PSA level was 5.5 ng/mL (FIG. 3). During follow-up, 29.9% of men developed biochemical recurrence, 11.4% of men developed metastatic lesions, and 5.0% of men died of prostate cancer (FIG. 3).

Aspn Aspartic Acid Repeat Length Variants and Prostate Cancer Incidence.

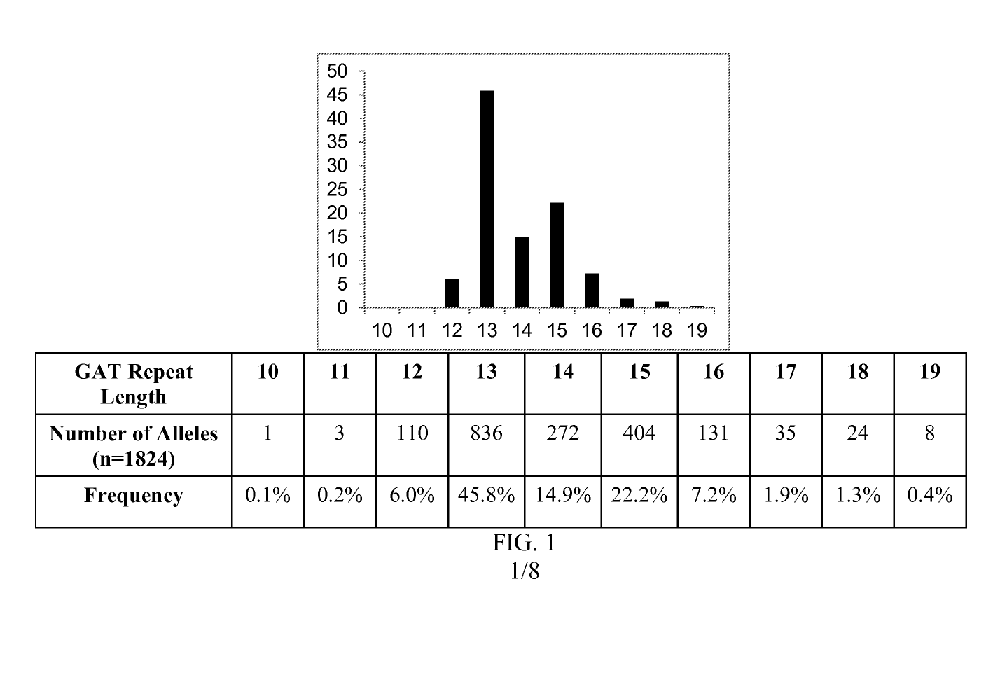

Men in this study had ASPND repeat lengths ranging from 10 to 19 aspartate residues with the most common lengths of 13 (45.8%), 15 (22.2%), 14 (14.9%), 16 (7.2%), and 12 (6.0%) residues. The least common aspartate residue lengths of 10, 11, 17, 18, and 19 were each less than 2% of the total (FIG. 1). This distribution accurately reflects that found in non-osteoarthritis control cohorts of US, UK, and Spanish Caucasian men and women as describe in Atif et al., 2008 (22), Mustafa et al., 2005 (23) and Rodriguez-Lopez et al., 2006 (24), respectively; suggesting that variations in ASPN D repeat length are not associated with prostate cancer incidence.

Aspn Aspartic Acid Repeat Length Variants and Metastatic Prostate Cancer.

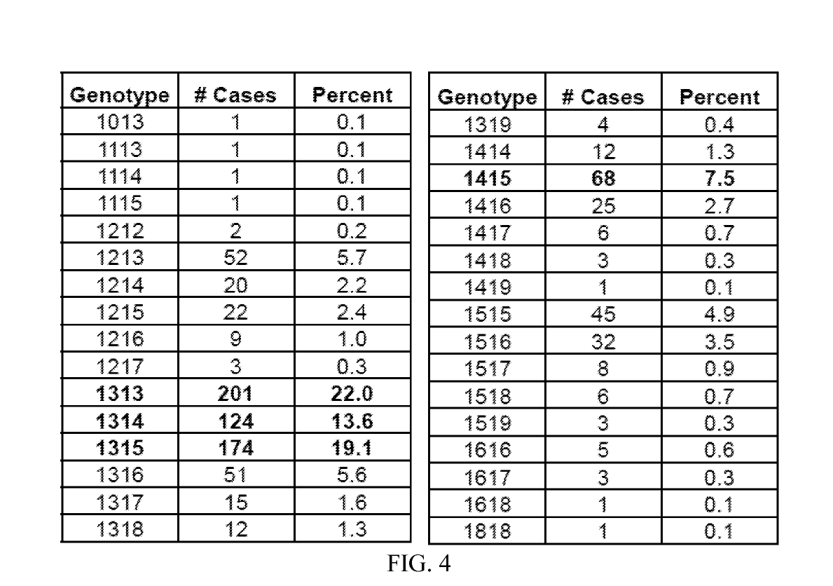

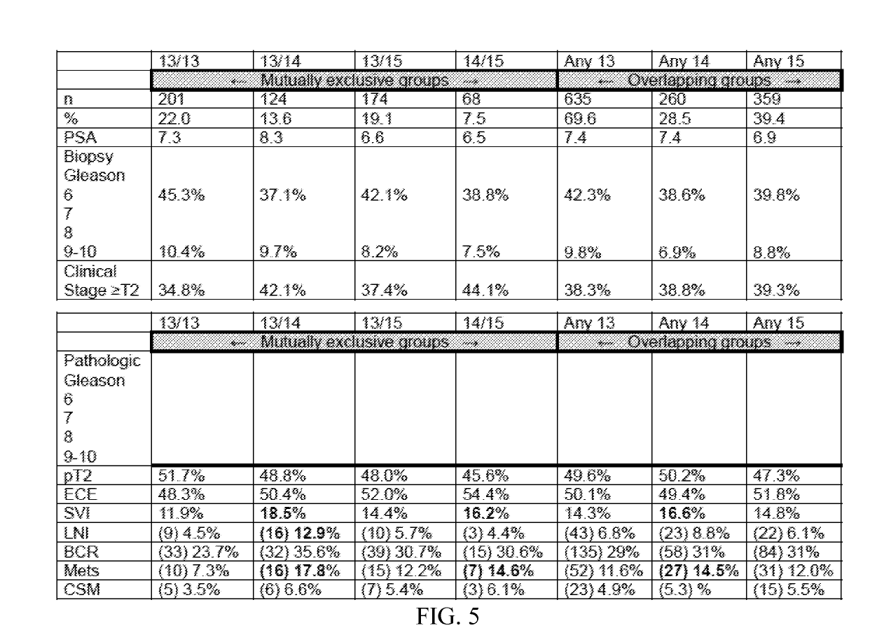

Pre-surgical nomagrams including PSA, biopsy Gleason grade and clinical stage are used to estimate the probabilities of pathological stage at surgery (25) and biochemical recurrence following surgery (26). Thus we examined for an association between ASPN D repeat length and pre-surgical factors. The most common ASPN D length genotypes in this cohort were 13/13 (22.0%), 13/15 (19.1%), 13/14 (13.6%), and 14/15 (7.5%) with the remaining genotypes between 0.1% and 5.7% of the study population (FIG. 4). To allow for adequately-powered comparative tests, genotypes present in ≧5% of the study population were selected for further analysis (where the integer corresponds to the number of aspartate-coding repeats in a single allele of ASPN): 13/13, 13/14, 13/15, and 14/15. Furthermore, single repeat length alleles present in ≧10% of the study population were also analyzed: any 13, any 14, and any 15. When compared to pre-surgical variables, none of the genotypes examined were associated with biopsy Gleason grade, PSA or clinical stage, suggesting that ASPN D length variants are not differentially associated with localized tumor growth or loss of differentiation (FIG. 5).

Post-surgical nomagrams including PSA, Gleason score and pathological stage are also used to estimate biochemical recurrence following surgery (26). Similar to pre-operative Gleason score from biopsy, ASPN D length variants were also not associated with pathological Gleason grade at surgery. ASPN D length variants were also not associated with local invasion through the prostatic capsule (extracapsular extension [ECE]) or to the seminal vesicles (seminal vesicle invasion [SVI]). In addition, none of the ASPN D repeat length variants were significantly associated with biochemical recurrence following surgery.

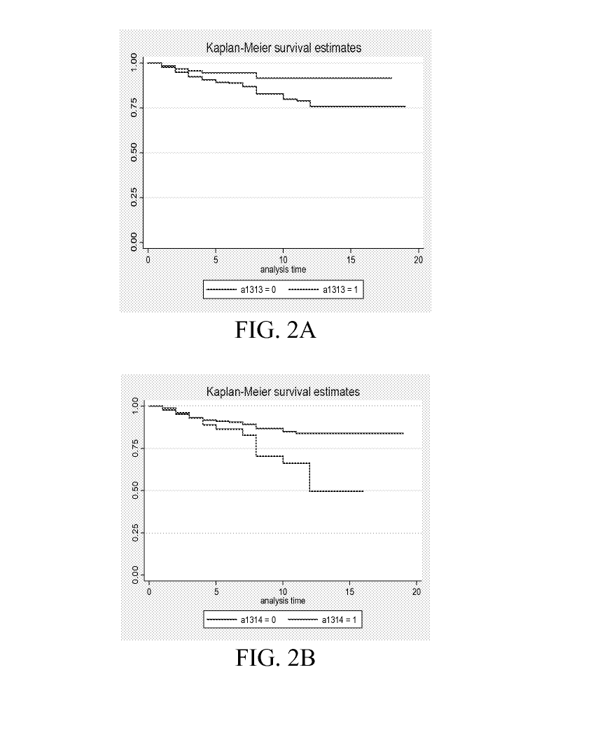

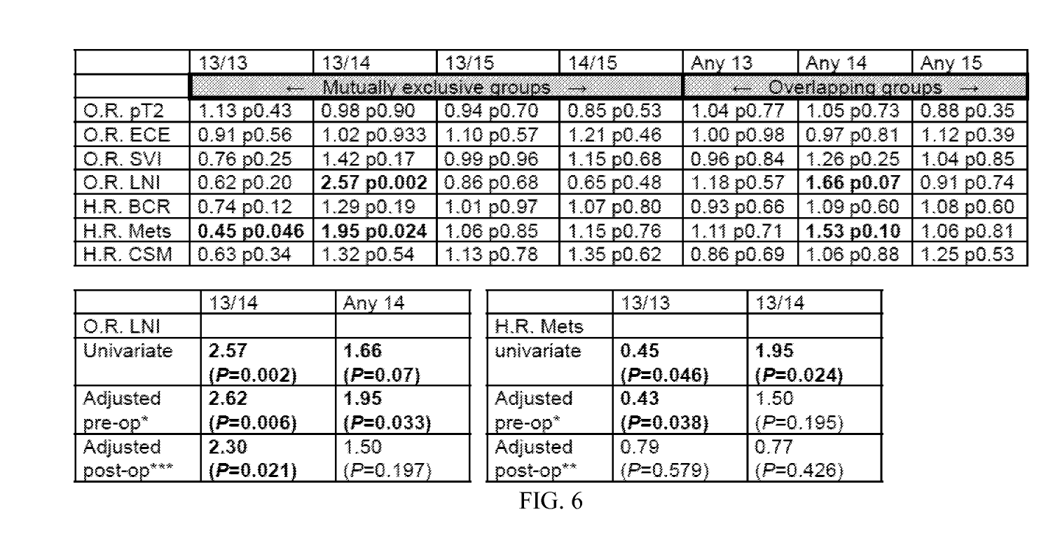

In contrast to localized invasion and biochemical recurrence, two common allelic variants were significantly associated, one negatively and one positively, with metastatic tumor progression. Multivariable Cox regression analyses adjusted for pre-operative variables demonstrated that ASPN D13/13 was significantly associated with a reduced risk of metastatic progression following surgery (HR: 0.45; 95% CI; P=0.038) (FIG. 6). When adjusted for post-operative variables, ASPN D13/13 was still associated with a reduced risk of metastatic progression but lost significance (FIG. 6). Kaplan-Meier survival estimates support that homozygous ASPN D repeat length of 13 is a significant germline marker prognostic of survival from metastatic prostate cancer (FIG. 2). ASPN D13/13 is associated with a medium time to progression of xxx years compared to xxx years for other allelic lengths. This study supports that homozygous carriers of ASPN D repeat length of 13 are less than half as likely to have metastatic prostate cancer recurrence following surgery than men with other common allelic D repeat lengths. Heterozygous carriers of ASPN D13, however, are not protected from metastatic progression suggesting that ASPN D13 is recessive to other allelic D repeat length variants.

While homozygous ASP N D13 is protective of metastatic prostate cancer recurrence, ASPN D14 is associated with metastatic progression. Multivariable Cox regression analyses adjusted for pre-operative variables demonstrated that men with ASPN D13/14 or carrying any ASPN D repeat length of 14 (homozygous or heterozygous) were significantly more likely to have lymph node metastases at surgery (OR for 13/14: 2.62; 95% CI, P=0.006 and OR for 14/x: 1.95; 95% CI, P=0.033) (FIG. 6). Kaplan-Meier survival estimates demonstrate heterozygous ASPN D13/14 is a significant germline prognostic marker of metastatic prostate cancer recurrence (FIG. 2). For metastatic disease, ASPN D13/14 was associated with a medium time to progression of xx years compared to men with other ASPN D genotypes. These data suggest that men with ASPN D13/14 are approximately twice as likely to have metastatic disease at surgery as men with other common ASPN D repeat lengths.

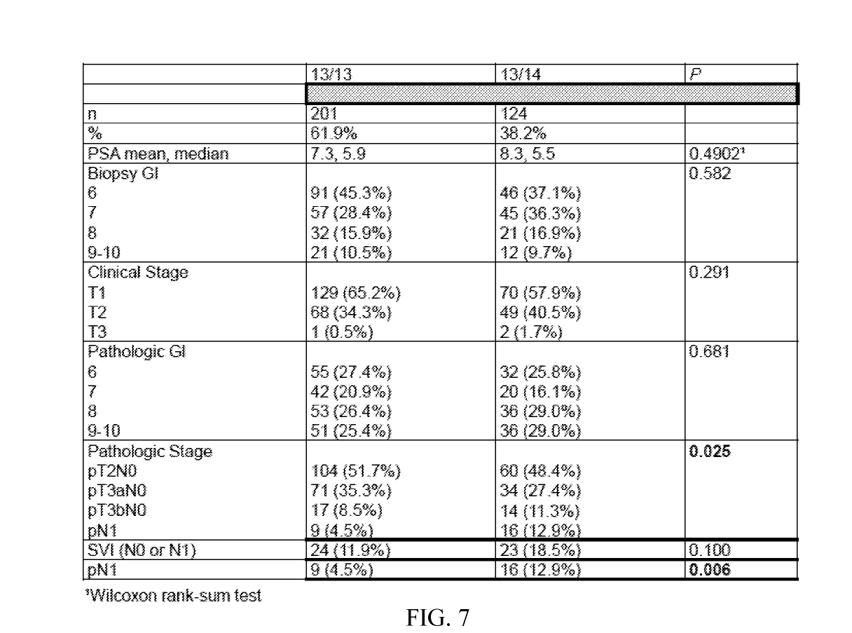

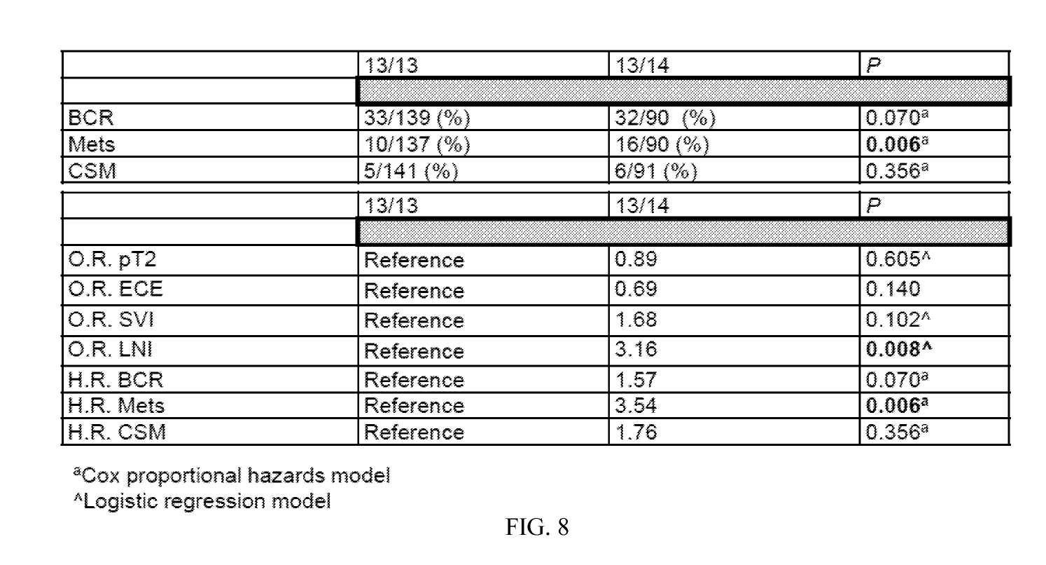

Over a third of Caucasian men are carriers of either homozygous ASPN D13 or heterozygous ASPN D13/14. When compared to ASPN D13/13, ASPN D13/14 was not associated with increases in PSA, biopsy Gleason grade, clinical stage, pathological Gleason grade, ECE or SVI (FIG. 7). However, multivariable analyses demonstrated that carriers of ASPN D13/14 were over three times as likely to have lymph node metastases at surgery (OR 3.16, P=0.008) and more than three and a half times as likely to have metastatic recurrence (HR 3.54, P=0.006) than men homozygous for ASPN D13 (FIG. 8).

### Discussion

Targeted analysis of germline ASPN D repeat domain length was analyzed in a cohort of patients treated with radical prostatectomy for prostate cancer at JHH. These analyses demonstrated that homozygous ASPN D13/13 is significantly protective of metastatic prostate cancer following surgery, while ASPN D14 (either homozygous or heterozygous) is significantly prognostic of lymph node metastases at surgery. Interestingly, heterozygous carriers of ASPN D13/x are not protected from prostate cancer metastases suggesting that potential protective functions of ASPN D13 are recessive to other alleles, especially ASPN D14. These findings have potential to impact therapeutic decisions in the clinic.

Controversy exists pertaining to the overtreatment of men with very low and low risk prostate cancer. Due to the indolent nature of these cancers, Active Surveillance is a treatment option for men with very low and low risk prostate cancer as defined in the NCCN guidelines (NCCN guidelines Prostate Cancer Version 2.2013). Despite this conservative option, many patients with very low to low risk prostate cancer undergo surgical or radiotherapeutic intervention, which are not without associated side effects and costs. Concerns over diagnostic accuracy of cancer aggressiveness often influence decisions towards definitive curative therapy. The development of a minimally-invasive and cost-effective genomic test for the early identification of aggressive prostate cancer has the potential to better identify those who do indeed need therapeutic intervention and as a corollary to spare thousands of men with indolent disease unnecessary surgical treatment.

Significant progress has been made in the past several years to identify genetic risk factors for prostate cancer. Genome wide association studies (GWAS) have identified several single nucleotide polymorphisms (SNP) associated with prostate cancer incidence (27-35); yet inherited determinants of aggressive prostate cancer have remained elusive. In this study, we demonstrate that while ASPN D repeat length variants do not affect the risk of being diagnosed with prostate cancer, two common variants affect the risk of having aggressive disease. While further studies and prospective trials are needed, this initial study suggests that inclusion of a genetic test for ASPN D repeat length may have clinical utility for men considering Active Surveillance as a treatment option. Due to its significantly protective association, homozygous ASPN D13 carriers may be better candidates for Active Surveillance than men with other allelic length variants. In contrast, due to its significant association with metastatic disease, carriers of ASPN D14 (either homozygous or heterozygous) may be better candidates for surgical intervention and possibly multi-modal therapies. As both polymorphisms in the D repeat domain length are common in Caucasian men, inclusion of these criteria for better delineation between indolent and aggressive prostate cancer has the potential to impact a substantial fraction of men considering Active Surveillance.

ASPN is an extracellular matrix (ECM) protein that has been shown to be elevated during androgen-induced early prostate development (1) and in cancer associated fibroblasts (8). Both its stromal specific expression in prostate cancer and its extracellular localization suggest that ASPN may have a role in modifying the ECM environment. One could postulate that the ASPN D13 allelic variant

Due to divergent allelic associations with metastatic prostate cancer, ASPN variants may differentially regulate processes driving metastatic progression.

Several studies suggest that ASPN may play an important role in modifying the ECM environment. The ASPN D14 polymorphism is associated with susceptibility to bone and joint diseases in Asian populations including osteoarthritis (12, 13, 15, 16), lumbar-disc degeneration (17), and developmental dysplasia of the hip (18). This, combined with our finding that ASPN D14 is also associated with metastatic prostate cancer, suggests that ASPN D polymorphisms may differentially regulate pathways common to both degenerative diseases and metastatic progression. It has been postulated that ASPN and in particular ASPN D14 inhibits TGFβ1 mediated chondrocyte differentiation and cartilage ECM formation (13). While the D repeat domain is not necessary for TGFβ1 binding (36), polymorphisms in the length of the D repeat domain have been shown to differentially affect the ability of ASPN to inhibit in vitro TGFβ1 induced cartilage matrix gene expression.

ASPN D14 has enhanced inhibition of TGFβ1-induced signaling over ASPN D13 while ASPN D16 and D17 variants do not inhibit TGFβ1 signaling (13). In addition to TGFβ1, ASPN has also been shown to inhibit other TGF family members such as BMP2 (37); yet the role of the ASPN aspartate domain length variants in BMP2 inhibition is not known. Polymorphisms in the aspartate repeat domain may differentially confer susceptibility to TGF family member mediated diseases, including prostate cancer. However, a role for ASPN in TGFβ1 or BMP2 signaling in the prostate is not known.

In addition to functioning through TGFβ, ASPN may directly modify the ECM through collagen interactions. ASPN has recently been shown to regulate osteoblast-driven collagen mineralization (19). While the ASPN N-terminal D repeat domain has been shown to bind calcium (19), the C-terminal domain of ASPN containing 10 Leucine Rich Repeats has been shown to bind to Type I (19) and to Type II (36) collagens. ASPN competes with Decorin, an inhibitor of prostate tumor growth (38), for collagen binding (19, 39, 40). In contrast to ASPN expression which is increased (8), Decorin expression is decreased in prostate cancer associated fibroblasts (41). ASPN and Decorin may work antagonistically to regulate the ECM environment. The differential roles of ASPN D length variants play in these processes have not been examined. Furthermore, the biologic and molecular mechanisms by which ASPN D14 may promote and ASPN D13/13 may inhibit metastatic prostate cancer are not known.

### REFERENCES

1. Schaeffer E M, Marchionni L, Huang Z, et al. Androgen-induced programs for prostate epithelial growth and invasion arise in embryogenesis and are reactivated in cancer. Oncogene 2008; 27: 7180-91.
2. Pritchard C, Mecham B, Dumpit R, et al. Conserved gene expression programs integrate mammalian prostate development and tumorigenesis. Cancer Res 2009; 69: 1739-47.
3. Hurley P J, Marchionni L, Simons B W, et al. Secreted protein, acidic and rich in cysteine-like 1 (SPARCL1) is down regulated in aggressive prostate cancers and is prognostic for poor clinical outcome. Proc Natl Acad Sci USA; 109: 14977-82.
4. Castellana B, Escuin D, Peiro G, et al. ASPN and GJB2 Are Implicated in the Mechanisms of Invasion of Ductal Breast Carcinomas. J Cancer; 3: 175-83.
5. Chou C H, Lee C H, Lu L S, et al. Direct assessment of articular cartilage and underlying subchondral bone reveals a progressive gene expression change in human osteoarthritic knees. Osteoarthritis Cartilage; 21: 450-61.
6. Gruber H E, Ingram J A, Hoelscher G L, Zinchenko N, Hanley E N, Jr., Sun Y. Asporin, a susceptibility gene in osteoarthritis, is expressed at higher levels in the more degenerate human intervertebral disc. Arthritis Res Ther 2009; 11: R47.
7. Klee E W, Bondar O P, Goodmanson M K, et al. Candidate serum biomarkers for prostate adenocarcinoma identified by mRNA differences in prostate tissue and verified with protein measurements in tissue and blood. Clin Chem; 58: 599-609.
8. Orr B, Riddick A C, Stewart G D, et al. Identification of stromally expressed molecules in the prostate by tag-profiling of cancer-associated fibroblasts, normal fibroblasts and fetal prostate. Oncogene; 31: 1130-42.
9. Turashvili G, Bouchal J, Baumforth K, et al. Novel markers for differentiation of lobular and ductal invasive breast carcinomas by laser microdissection and microarray analysis. BMC Cancer 2007; 7: 55.
10. Turtoi A, Musmeci D, Wang Y, et al. Identification of novel accessible proteins bearing diagnostic and therapeutic potential in human pancreatic ductal adenocarcinoma. J Proteome Res; 10: 4302-13.
11. Zhang R, Fang H, Chen Y, et al. Gene expression analyses of subchondral bone in early experimental osteoarthritis by microarray. PLoS One; 7: e32356.
12. Ikegawa S, Kawamura S, Takahashi A, Nakamura T, Kamatani N. Replication of association of the D-repeat polymorphism in asporin with osteoarthritis. Arthritis Res Ther 2006; 8: 403; author reply
13. Kizawa H, Kou I, Iida A, et al. An aspartic acid repeat polymorphism in asporin inhibits chondrogenesis and increases susceptibility to osteoarthritis. Nat Genet 2005; 37: 138-44.
14. Liu D, Yang Q, Li M, Mu K, Zhang Y. Association of an asporin repeat polymorphism with ankylosing spondylitis in Han Chinese population: a case-control study. Clin Invest Med; 33: E63-8.
15. Shi D, Nakamura T, Dai J, et al. Association of the aspartic acid-repeat polymorphism in the asporin gene with age at onset of knee osteoarthritis in Han Chinese population. J Hum Genet 2007; 52: 664-7.
16. Song J H, Lee H S, Kim C J, et al. Aspartic acid repeat polymorphism of the asporin gene with susceptibility to osteoarthritis of the knee in a Korean population. Knee 2008; 15: 191-5.
17. Song Y Q, Cheung K M, Ho D W, et al. Association of the asporin D14 allele with lumbar-disc degeneration in Asians. Am J Hum Genet 2008; 82: 744-7.
18. Shi D, Dai J, Zhu P, et al. Association of the D repeat polymorphism in the ASPN gene with developmental dysplasia of the hip: a case-control study in Han Chinese. Arthritis Res Ther; 13: R27.
19. Kalamajski S, Aspberg A, Lindblom K, Heinegard D, Oldberg A. Asporin competes with decorin for collagen binding, binds calcium and promotes osteoblast collagen mineralization. Biochem J 2009; 423: 53-9.
20. Epstein J I, Pizov G, Walsh P C. Correlation of pathologic findings with progression after radical retropubic prostatectomy. Cancer 1993; 71: 3582-93.
21. Sun J, Purcell L, Gao Z, et al. Association between sequence variants at 17q12 and 17q24.3 and prostate cancer risk in European and African Americans. Prostate 2008; 68: 691-7.
22. Atif U, Philip A, Aponte J, et al. Absence of association of asporin polymorphisms and osteoarthritis susceptibility in US Caucasians. Osteoarthritis Cartilage 2008; 16: 1174-7.
23. Mustafa Z, Dowling B, Chapman K, Sinsheimer J S, Carr A, Loughlin J. Investigating the aspartic acid (D) repeat of asporin as a risk factor for osteoarthritis in a UK Caucasian population. Arthritis Rheum 2005; 52: 3502-6.
24. Rodriguez-Lopez J, Pombo-Suarez M, Liz M, Gomez-Reino J J, Gonzalez A. Lack of association of a variable number of aspartic acid residues in the asporin gene with osteoarthritis susceptibility: case-control studies in Spanish Caucasians. Arthritis Res Ther 2006; 8: R55.
25. Eifler J B, Feng Z, Lin B M, et al. An updated prostate cancer staging nomogram (Partin tables) based on cases from 2006 to 2011. BJU Int; 111: 22-9.
26. Han M, Partin A W, Zahurak M, Piantadosi S, Epstein J I, Walsh P C. Biochemical (prostate specific antigen) recurrence probability following radical prostatectomy for clinically localized prostate cancer. J Urol 2003; 169: 517-23.
27. Maclnnis R J, Severi G, Baglietto L, et al. Population-based estimate of prostate cancer risk for carriers of the HOXB13 missense mutation G84E. PLoS One; 8: e54727.
28. Xu J, Lange E M, Lu L, et al. HOXB13 is a susceptibility gene for prostate cancer: results from the International Consortium for Prostate Cancer Genetics (ICPCG). Hum Genet; 132: 5-14.
29. Ewing C M, Ray A M, Lange E M, et al. Germline mutations in HOXB13 and prostate-cancer risk. N Engl J Med; 366: 141-9.
30. Shang Z, Zhu S, Zhang H, Li L, Niu Y. Germline Homeobox B13 (HOXB13) G84E Mutation and Prostate Cancer Risk in European Descendants: A Meta-analysis of 24 213 Cases and 73 631 Controls. Eur Urol.
31. Witte J S, Mefford J, Plummer S J, et al. HOXB13 mutation and prostate cancer: studies of siblings and aggressive disease. Cancer Epidemiol Biomarkers Prev; 22: 675-80.
32. Stott-Miller M, Karyadi D M, Smith T, et al. HOXB13 mutations in a population-based, case-control study of prostate cancer. Prostate; 73: 634-41.
33. Kote-Jarai Z, Easton D F, Stanford J L, et al. Multiple novel prostate cancer predisposition loci confirmed by an international study: the PRACTICAL Consortium. Cancer Epidemiol Biomarkers Prev 2008; 17: 2052-61.
34. Eeles R A, Kote-Jarai Z, Giles G G, et al. Multiple newly identified loci associated with prostate cancer susceptibility. Nat Genet 2008; 40: 316-21.
35. Thomas G, Jacobs K B, Yeager M, et al. Multiple loci identified in a genome-wide association study of prostate cancer. Nat Genet 2008; 40: 310-5.
36. Kou I, Nakajima M, Ikegawa S. Binding characteristics of the osteoarthritis-associated protein asporin. J Bone Miner Metab; 28: 395-402.
37. Tomoeda M, Yamada S, Shirai H, Ozawa Y, Yanagita M, Murakami S. PLAP-1/asporin inhibits activation of BMP receptor via its leucine-rich repeat motif Biochem Biophys Res Commun 2008; 371: 191-6.
38. Hu Y, Sun H, Owens R T, et al. Decorin suppresses prostate tumor growth through inhibition of epidermal growth factor and androgen receptor pathways. Neoplasia 2009; 11: 1042-53.
39. Lorenzo P, Aspberg A, Onnerfjord P, Bayliss M T, Neame P J, Heinegard D. Identification and characterization of asporin. a novel member of the leucine-rich repeat protein family closely related to decorin and biglycan. J Biol Chem 2001; 276: 12201-11.
40. Henry S P, Takanosu M, Boyd T C, et al. Expression pattern and gene characterization of asporin. a newly discovered member of the leucine-rich repeat protein family. J Biol Chem 2001; 276: 12212-21.
41. Henke A, Grace O C, Ashley G R, et al. Stromal expression of decorin, Semaphorin6D, SPARC, Sproutyl and Tsukushi in developing prostate and decreased levels of decorin in prostate cancer. PLoS One; 7: e42516.

1. A method for determining a likelihood of prostate cancer recurrence in a patient following prostectomy comprising the steps of:
a. obtaining a biological sample from the patient;
b. subjecting the sample to an assay for genotyping the aspartic acid (D) repeat polymorphism located in the N-terminal region of the Asporin (ASPN) gene;
c. determining that prostate cancer is less likely to recur if the ASPN D repeat domain length is 13 in both alleles as compared to other allelic genotypes; and
d. determining that prostate cancer is more likely to recur if the ASPN D repeat domain length is 13 in one allele and 14 in the other allele or have at least one allele with 14 D repeats as compared to other allelic genotypes.

2. A method for predicting metastasis in prostate cancer patient comprising the steps of:
a. obtaining a biological sample from the patient;
b. subjecting the sample to an assay for genotyping the aspartic acid (D) repeat polymorphism located in the N-terminal region of the Asporin (ASPN) gene;
c. determining that metastasis is less likely to occur if the ASPN D repeat domain length is 13 in both alleles as compared to other allelic genotypes; and
d. determining that metastasis is more likely to recur if the ASPN D repeat domain length is 13 in one allele and 14 in the other allele or have at least one allele with 14 D repeats as compared to other allelic genotypes.

3. A method for identifying prostate cancer lesions with metastatic potential in a patient comprising the steps of:
a. obtaining a biological sample from the patient;
b. subjecting the sample to an assay for genotyping the aspartic acid (D) repeat polymorphism located in the N-terminal region of the Asporin (ASPN) gene;
c. determining that the prostate cancer lesions have lower metastatic potential if the ASPN D repeat domain length is 13 in both alleles as compared to other allelic genotypes; and
d. determining that the prostate cancer lesions have higher metastatic potential if the ASPN D repeat domain length is 13 in one allele and 14 in the other allele or have at least one allele with 14 D repeats as compared to other allelic genotypes.

4. The method of claim 1, wherein the obtaining the biological sample step comprises obtaining tissue for genomic DNA isolation.

5. The method of claim 1, wherein the performing an assay step comprises performing a polymerase chain reaction.

6. The method of claim 5, wherein the polymerase chain reaction is performed using the primer described in SEQ ID NO:1 and SEQ ID NO:2.

7. A method for predicting metastasis in a prostate cancer patient comprising the steps of:
a. genotyping the ASPN D repeat domain length in both alleles of the patient using a polymerase chain reaction;
b. predicting metastasis in the patient if the ASPND repeat domain length is 13 in one allele and 14 in the other allele or have at least one allele with 14 D repeats as compared to other common allelic genotypes; and
c. predicting no metastasis in the patient if the ASPN D repeat domain length is 13 in both alleles as compared to other allelic genotypes.
